(19) United States
(12) Patent Application Publication
(10) Pub. No.: US 2024/0366061 A1
(43) Pub. Date: Nov. 7, 2024

(54) ENDOSCOPE SYSTEM, METHOD FOR CONTROLLING ENDOSCOPE SYSTEM, AND RECORDING MEDIUM

(71) Applicants: OLYMPUS CORPORATION, Tokyo (JP); National Cancer Center, Tokyo (JP)

(72) Inventors: Hiroyuki TAKAYAMA, Tokyo (JP); Chiharu MIZUTANI, Tokyo (JP); Hiroto OGIMOTO, Tokyo (JP); Masaaki ITO, Tokyo (JP); Hiro HASEGAWA, Tokyo (JP); Daichi KITAGUCHI, Tokyo (JP); Yuki FURUSAWA, Tokyo (JP)

(73) Assignees: OLYMPUS CORPORATION, Tokyo (JP); National Cancer Center, Tokyo (JP)

(21) Appl. No.: 18/777,636

(22) Filed: Jul. 19, 2024

Related U.S. Application Data
(63) Continuation of application No. PCT/JP2022/045971, filed on Dec. 14, 2022.
(60) Provisional application No. 63/303,158, filed on Jan. 26, 2022.

(51) Int. Cl. A61B 1/00

(52) U.S. Cl. CPC A61B 1/00009; A61B 1/00006; A61B 1/00042; A61B 1/00149

(57) ABSTRACT

An endoscope system includes an endoscope to be inserted into a subject to acquire an image, a robot arm that is configured to change the position and the posture of the endoscope, and a controller including a processor. The controller is configured to: acquire treatment instrument information concerning the position or the movement of a treatment instrument to be inserted into the subject, determine whether the treatment instrument has been removed based on the treatment instrument information, in response to determining that the treatment instrument has been removed, execute an overlooking mode, and in the overlooking mode, control at least one of the endoscope or the robot arm to thereby automatically zoom out the image while maintaining, in the image, a specific point in the subject.

### CROSS-REFERENCE TO RELATED APPLICATIONS

This is a continuation of International Application PCT/JP2022/045971, with an international filing date of Dec. 14, 2022, which is hereby incorporated by reference herein in its entirety.

This application claims the benefit of U.S. Provisional Application No. 63/303,158, filed Jan. 26, 2022, which is hereby incorporated by reference herein in its entirety. The present disclosure relates to an endoscope system, a method for controlling the endoscope system, and a recording medium.

### BACKGROUND

There has been proposed a system that controls a robot arm based on the position of a treatment instrument to thereby cause an endoscope to automatically follow the treatment instrument (see, for example, PTL 1). The system of PTL 1 controls the robot arm to continuously capture the treatment instrument in the center of an image of the endoscope.

### CITATION LIST

### Patent Literature

{PTL 1} Japanese Unexamined Patent Application, Publication No. 2003-127076.

### SUMMARY

The present disclosure has been made in view of the circumstances explained above, and an object of the present disclosure is to provide an endoscope system, a control method for the endoscope system, and a recording medium that can support easy reinsertion of a treatment instrument without requiring operation of a user.

### SOLUTION TO PROBLEM

An aspect of the present disclosure is an endoscope system comprising: an endoscope to be inserted into a subject to acquire an image of an inside of the subject; a robot arm configured to change a position and a posture of the endoscope; and a control device comprising at least one processor, wherein the control device is configured to: acquire treatment instrument information concerning a position or a movement of a treatment instrument to be inserted into the subject, determine whether the treatment instrument has been removed based on the treatment instrument information, in response to determining that the treatment instrument has been removed, execute an overlooking mode, and, in the overlooking mode, control at least one of the endoscope or the robot arm to thereby automatically zoom out the image while maintaining, in the image, a specific point in the subject.

Another aspect of the present disclosure is a method for controlling an endoscope system, the endoscope system comprising: an endoscope to be inserted into a subject to acquire an image of an inside of the subject; and a robot arm configured to change a position and a posture of the endoscope, the control method comprising: acquiring treatment instrument information concerning a position or a movement of a treatment instrument inserted into the subject; determining whether the treatment instrument has been removed based on the treatment instrument information; in response to determining that the treatment instrument has been removed, executing an overlooking mode; and, in the overlooking mode, controlling at least one of the endoscope or the robot arm to thereby automatically zoom out the image while maintaining, in the image, a specific point in the subject.

Another aspect of the present disclosure is a non-transitory computer-readable recording medium storing a control program for causing a computer to execute the control method described above.

### BRIEF DESCRIPTION OF DRAWINGS

FIG. 1 is an overall configuration diagram of an endoscope system according to an embodiment.

FIG. 2 is a block diagram illustrating an overall configuration of the endoscope system illustrated in FIG. 1.

FIG. 3A is a diagram for explaining an operation of an endoscope in a following mode or a manual mode.

FIG. 3B is a diagram for explaining an operation of the endoscope in the following mode or the manual mode.

FIG. 3C is a diagram for explaining an operation of the endoscope in an overlooking mode.

FIG. 3D is a diagram for explaining an operation of the endoscope in the overlooking mode.

FIG. 3E is a diagram for explaining an operation of the endoscope in the overlooking mode.

FIG. 4A is a diagram illustrating an example of an image during the following mode or the manual mode.

FIG. 4B is a diagram illustrating an example of an image during the following mode or the manual mode after a treatment instrument is removed.

FIG. 4C is a diagram illustrating an example of a zoomed-out image during the overlooking mode.

FIG. 5 is a flowchart of a control method for the endoscope system.

FIG. 6A is a flowchart of a first method for determining a start trigger.

FIG. 6B is a flowchart of a second method for determining the start trigger.

FIG. 6C is a flowchart of a third method for determining the start trigger.

FIG. 6D is a flowchart of a fourth method for determining the start trigger.

FIG. 7 is a flowchart of a method of determining an end trigger.

FIG. 8 is a diagram illustrating a zoomed-out image in which an inner wall of a trocar is reflected.

FIG. 9A is a diagram illustrating an example of a zoomed-out image in which a reinserted treatment instrument is reflected.

FIG. 9B is a diagram illustrating another example of the zoomed-out image in which the reinserted treatment instrument is reflected.

FIG. 10A is a diagram illustrating an example of a zoomed-out image after movement of a visual field of the endoscope.

FIG. 10B is a diagram illustrating another example of the zoomed-out image after the movement of the visual field of the endoscope.

FIG. 11 is a flowchart of a modification of the method of determining the end trigger.

### DESCRIPTION OF EMBODIMENTS

An endoscope system, a method for controlling the endoscope system, and a recording medium according to an embodiment of the present disclosure are explained below with reference to the drawings.

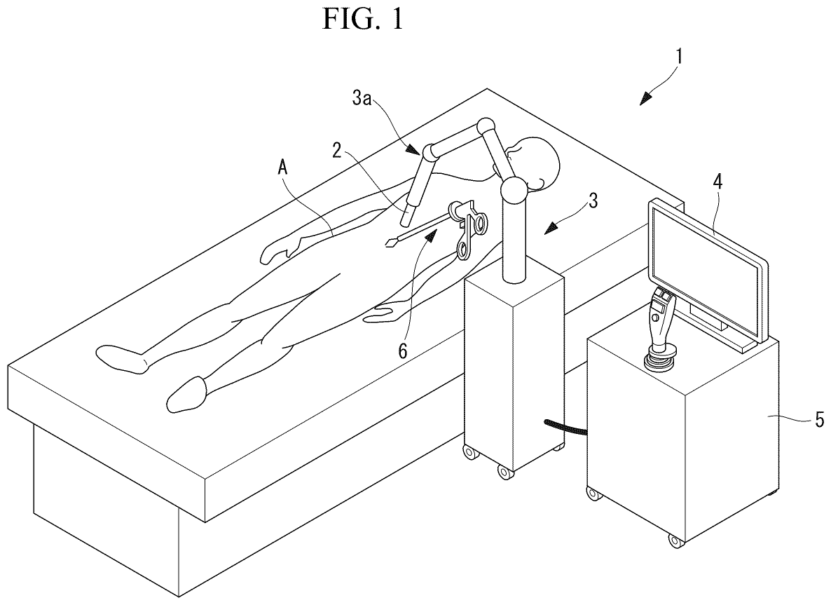

As illustrated in FIG. 1, an endoscope system 1 according to the present embodiment is used for surgery for inserting an endoscope 2 and a treatment instrument 6 into a body of a patient, who is a subject A, and treating a target site such as a diseased part with the treatment instrument 6 while observing the treatment instrument 6 with the endoscope 2 and is used for, for example, laparoscopic surgery. The treatment instrument 6 is an energy treatment instrument that performs tissue dissection and pealing, blood vessel sealing, or the like with, for example, a high-frequency current or ultrasonic vibration or is forceps for gripping a tissue. However, an example explained above is only an example and, not only this, but various treatment instruments generally used in endoscopic surgery may be used.

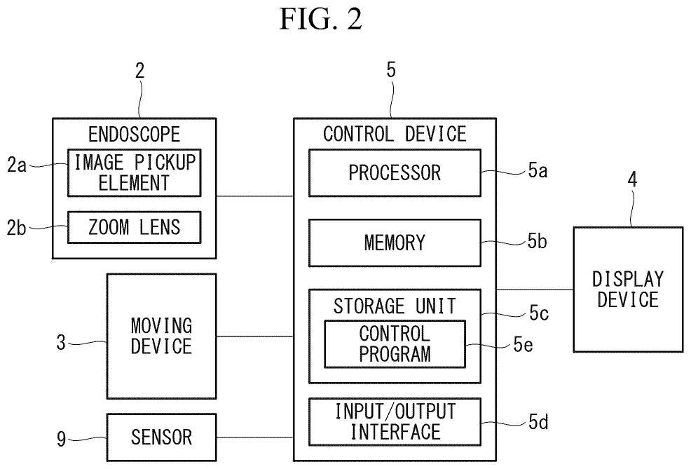

As illustrated in FIG. 1 and FIG. 2, the endoscope system 1 includes the endoscope 2 to be inserted into the subject A, a moving device 3 that moves the endoscope 2, a display device 4, and a control device (controller) 5 that controls the endoscope 2 and the moving device 3.

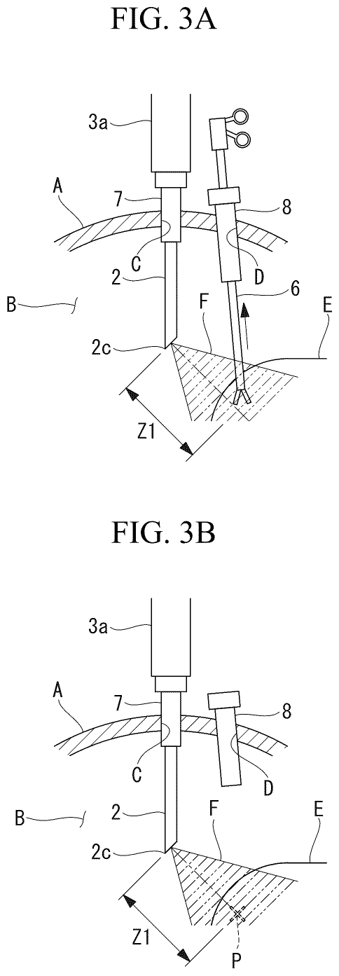

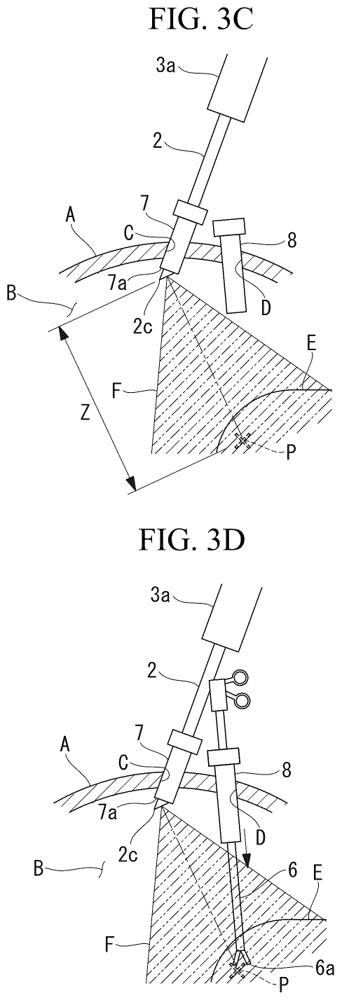

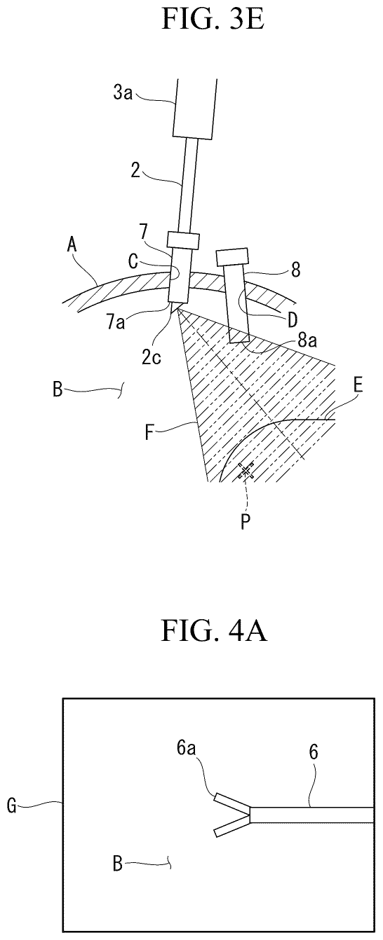

As illustrated in FIG. 3A to FIG. 3D, the endoscope 2 and the treatment instrument 6 are inserted into the subject A, for example, an abdominal cavity B respectively through trocars 7 and 8. The trocars 7 and 8 are tubular equipment opened at both ends. The trocars 7 and 8 respectively pierce through holes C and D formed in a body wall and is capable of swinging with the positions of the holes C and D, which are pivot points, as fulcrums.

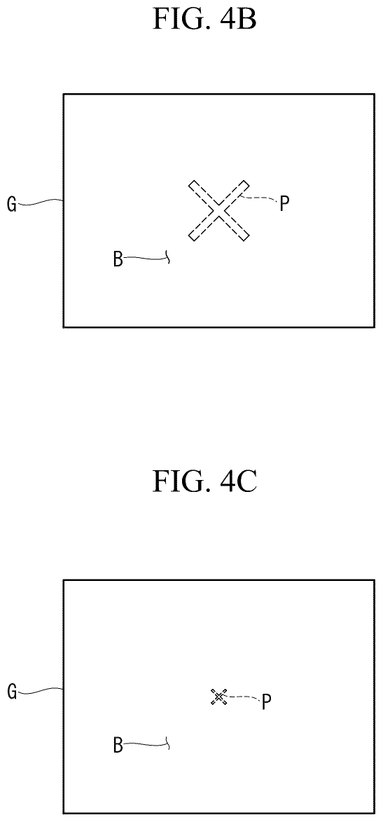

The endoscope 2 is, for example, an oblique-view type rigid scope. The endoscope 2 may be a forward-view type. The endoscope 2 includes an image pickup element 2a such as a CCD image sensor or a CMOS image sensor and acquires an image G (see FIG. 4A to FIG. 4C) in the subject A. The image pickup element 2a is, for example, a three-dimensional camera provided at the distal end portion of the endoscope 2 and picks up a stereoscopic image as the image G. An objective lens of the endoscope 2 may include a zoom lens 2b that optically changes the magnification of the image G.

The image G is transmitted from the endoscope 2 to the display device 4 through the control device 5 and displayed on the display device 4. The display device 4 is any display such as a liquid crystal display or an organic EL display.

The moving device 3 includes an electric holder 3a including an articulated robot arm and is controlled by the control device 5. The endoscope 2 is held at the distal end portion of the electric holder 3a. The position and the posture of the endoscope 2 are three-dimensionally changed by a motion of the electric holder 3a. Note that the moving device 3 does not always need to be separate from the endoscope 2 and may be integrally formed as a part of the endoscope 2.

The control device 5 is an endoscope processor that controls the endoscope 2, the moving device 3, and the image G displayed on the display device 4. As illustrated in FIG. 2, the control device 5 includes at least one processor 5a, a memory 5b, a storage unit 5c, and an input/output interface 5d.

The control device 5 is connected to peripheral equipment 2, 3, 4, and 9 through the input/output interface 5d and transmits and receives the image G, signals, and the like through the input/output interface 5d.

The storage unit 5c is a non-transitory computer-readable recording medium and is, for example, a hard disk drive, an optical disk, or a flash memory. The storage unit 5c stores a control program 5e for causing the processor 5a to execute a control method explained below and data necessary for processing of the processor 5a.

A part of processing explained below executed by the processor 5a may be implemented by a dedicated logical circuit, hardware, or the like such as an FPGA (Field Programmable Gate Array), an SoC (System-On-A-Chip), an ASIC (Application Specific Integrated Circuit), or a PLD (Programmable Logic Device).

The processor 5a controls at least one of the endoscope 2 or the moving device 3 in any one of a plurality of modes including a manual mode, a following mode, and an overlooking mode according to the control program 5e read from the storage unit 5c in the memory 5b such as a RAM (Random Access Memory). A user can select one of the manual mode and the following mode using a user interface (not illustrated) provided in the control device 5.

The manual mode is a mode for permitting operation of the endoscope 2 by the user such as a surgeon. In the manual mode, the user can remotely operate the endoscope 2 using a master device (not illustrated) connected to the moving device 3. For example, the master device includes input devices such as buttons, a joystick, and a touch panel. The processor 5a controls the moving device 3 according to a signal from the master device. The user may directly grip the proximal end portion of the endoscope 2 with a hand and manually move the endoscope 2.

The following mode is a mode in which the control device 5 causes the endoscope 2 to automatically follow the treatment instrument 6. In the following mode, the processor 5a recognizes the treatment instrument 6 in the image G using a publicly-known image recognition technique, acquires a three-dimensional position of a distal end 6a of the treatment instrument 6 through stereoscopic measurement using the image G, and controls the moving device 3 based on the three-dimensional position of the distal end 6a and a three-dimensional position of a predetermined target point. The target point is a point set in a visual field F of the endoscope 2 and is, for example, a point on an optical axis of the endoscope 2 separated from a distal end 2c of the endoscope 2 by a predetermined observation distance Z1. Accordingly, as illustrated in FIG. 4A, the control device 5 causes the endoscope 2 to follow the treatment instrument 6 such that the distal end 6a is disposed in the center of the image G.

The overlooking mode is a mode in which the control device 5 controls at least one of the endoscope 2 or the moving device 3 to thereby automatically zoom out the image G and overlooks the inside of the subject A. The processor 5a acquires treatment instrument information during the manual or following mode and automatically starts and ends the overlooking mode based on the treatment instrument information.

Subsequently, a control method executed by the processor 5a is explained.

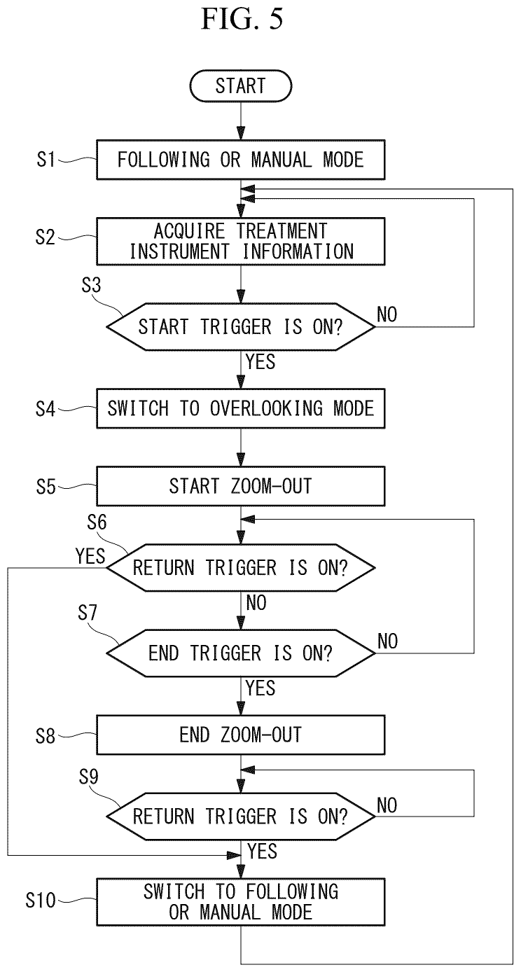

As illustrated in FIG. 5, the control method includes a step S1 of controlling the endoscope 2 and the moving device 3 in the manual mode or the following mode, a step S2 of acquiring treatment instrument information, a step S3 of determining a start trigger, a step S4 of switching the manual mode or the following mode to the overlooking mode, a step S5 of starting zoom-out, a step S7 of determining an end trigger, a step S8 of ending the zoom-out, steps S6 and S9 of determining a return trigger, and a step S10 of switching the overlooking mode to the manual mode or the following mode.

The processor 5a controls, based on input of the user to the user interface, the moving device 3 and the endoscope 2 in the following mode or the manual mode (step S1). As illustrated in FIG. 3A to FIG. 3D, during the following mode or the manual mode, for replacement or the like of the treatment instrument 6, the user removes the treatment instrument 6 from the inside of the subject A and thereafter reinserts the treatment instrument 6 into the subject A.

During the following mode or the manual mode, the processor 5a repeatedly acquires treatment instrument information concerning the position or the movement of the treatment instrument 6 (step S2). The processor 5a determines, based on the treatment instrument information, a start trigger indicating that the treatment instrument 6 has been removed (step S3) and starts the overlooking mode in response to the start trigger being turned on (step S4).

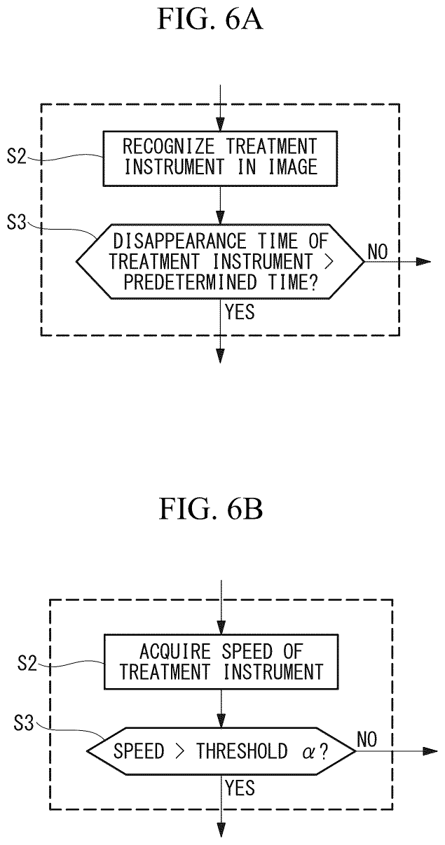

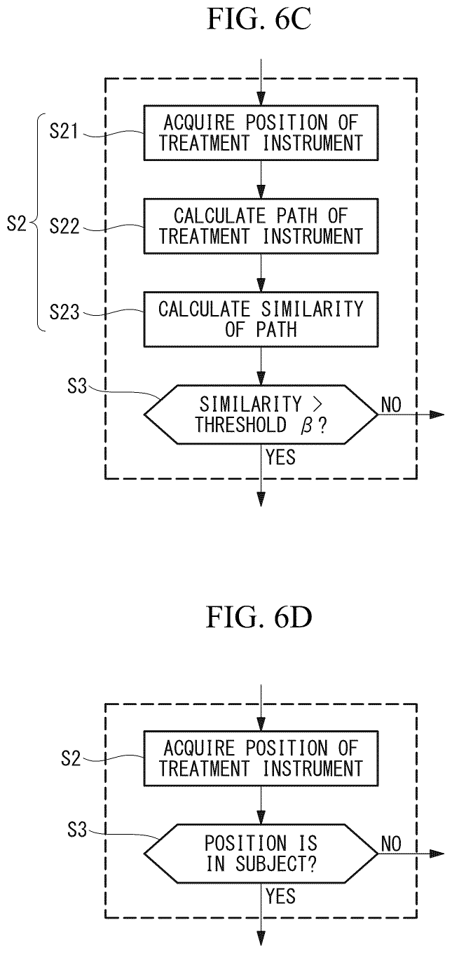

FIG. 6A to FIG. 6D explain an example of the determination of the start trigger.

In a first method illustrated in FIG. 6A, the treatment instrument information is presence or absence of the treatment instrument 6 in the image G. The processor 5a detects absence of the treatment instrument 6 in the image G as the start trigger. Specifically, the processor 5a recognizes the treatment instrument 6 in the image G using the publicly-known image recognition technique (step S2). When the treatment instrument 6 is present in the image G and has been recognized, the processor 5a determines that the start trigger is OFF (NO in step S3). When the treatment instrument 6 is absent in the image G and has not been recognized, the processor 5a determines that the start trigger is ON (YES in step S3).

The speed of the treatment instrument 6 at the removal time is higher compared with the speed of the endoscope 2 that follows the treatment instrument 6. Therefore, the treatment instrument 6 disappears from the image G halfway in a removing motion. The processor 5a temporarily stops the endoscope 2. Therefore, since the image G in which the treatment instrument 6 is absence is acquired after the removal, it is possible to detect the removal based on presence or absence of the treatment instrument 6 in the image G.

In the first method, the processor 5a preferably uses, as the treatment instrument information, a disappearance time in which the treatment instrument 6 is continuously absent in the image G. In this case, when the disappearance time is equal to or shorter than a predetermined time (a threshold), the processor 5a determines that the start trigger is OFF (NO in step S3). When the disappearance time has exceeded the predetermined time, the processor 5a determines that the start trigger is ON (YES in step S3).

The treatment instrument 6 sometimes disappears from the image G for a while regardless of the presence of the treatment instrument 6 in the subject A because the endoscope 2 cannot catch up with the treatment instrument 6 that is moving fast in the subject A. It is possible to more accurately detect the removal by determining whether the disappearance time has exceeded the predetermined time.

In a second method illustrated in FIG. 6B, the treatment instrument information is the speed of the treatment instrument 6. The processor 5a detects, as the start trigger, the speed of the treatment instrument 6 being larger than a predetermined speed threshold a. Specifically, the processor 5a acquires the speed of the treatment instrument 6 (step S2). When the speed is equal to or smaller than the threshold a, the processor 5a determines that the start trigger is OFF (NO in step S3). When the speed is larger than the threshold a, the processor 5a determines that the start trigger is ON (YES in step S3).

The speed of the treatment instrument 6 at the removal time is far higher than the speed of the treatment instrument 6 at time other than the removal time. Therefore, it is possible to accurately detect the removal of the treatment instrument 6 based on the speed.

In a third method illustrated in FIG. 6C, the treatment instrument information is a path on which the treatment instrument 6 has moved in the subject A. The processor 5a detects, as the start trigger, the treatment instrument 6 having moved along a predetermined path. Specifically, the processor 5a acquires the position of the treatment instrument 6 in the subject A (step S21), calculates a path of the treatment instrument 6 from the acquired position (step S22), and calculates similarity of the calculated path to the predetermined path (step S23). When the similarity is equal to or smaller than a predetermined similarity threshold B, the processor 5a determines that the start trigger is OFF (NO in step S3). When the similarity is larger than the threshold B, the processor 5a determines that the start trigger is ON (YES in step S3).

The treatment instrument 6 to be removed retracts along a predetermined path specified by the trocar 8. Therefore, it is possible to accurately detect the removal of the treatment instrument 6 based on the path of the treatment instrument 6.

In a fourth method illustrated in FIG. 6D, the treatment instrument information is the position of the treatment instrument 6. The processor 5a detects, as the start trigger, the position of the treatment instrument 6 being on the outer side of the subject A. Specifically, the processor 5a acquires the position of the treatment instrument 6 in a three-dimensional space including the inner side and the outer side of the subject A (step S21). When the position of the treatment instrument 6 is within the subject A, the processor 5a determines that the start trigger is OFF (NO in step S3). When the position of the treatment instrument 6 is outside the subject A, the processor 5a determines that the start trigger is ON (YES in step S3).

As explained above, the treatment instrument information includes the presence or absence of the treatment instrument 6 in the image G, the length of the disappearance time, and any one of the speed, the path, and the position of the treatment instrument 6. These kinds of treatment instrument information is detected using the image G or detected using any sensor 9 that detects a three-dimensional position of the treatment instrument 6.

The endoscope system 1 may further include a treatment instrument information detection unit that detects treatment instrument information. The treatment instrument information detection unit may be a processor that is provided in the control device 5 and detects the treatment instrument information from the image G. The processor may be the processor 5a or another processor. Alternatively, the treatment instrument information detection unit may be the sensor 9.

When determining that the start trigger is OFF (NO in step S3), the processor 5a repeats steps S2 and S3.

When determining that the start trigger is ON (YES in step S3), the processor 5a switches the following mode or the manual mode to the overlooking mode (step S4) and subsequently starts zoom-out of the image G (step S5).

In step S5, the processor 5a controls at least one of the moving device 3 or the endoscope 2 to thereby zoom out the image G while maintaining, in the image G, a specific point P in the subject A.

The specific point P is a position where a predetermined point is arranged in the visual field F at a point in time when it is determined that the start trigger is ON. For example, as illustrated in FIG. 3B, the specific point P is a position where a target point is arranged at the point in time when it is determined that the start trigger is ON. As illustrated in FIG. 4B, the specific point P is arranged in the center in the image G.

Specifically, in step S5, the processor 5a calculates a position coordinate of the specific point P in a world coordinate system, for example, from rotation angles of joints of a robot arm 3a and the observation distance Z1. The world coordinate system is a coordinate system fixed with respect to a space in which the endoscope system 1 is disposed. The world coordinate system is, for example, a coordinate system in which the proximal end of the robot arm 3a is the origin.

Subsequently, as illustrated in FIG. 3C, the processor 5a controls the moving device 3 to thereby retract the endoscope 2 and moves the distal end 2c of the endoscope 2 in a direction away from the specific point P while maintaining the specific point P on the optical axis. Accordingly, as illustrated in FIG. 4C, the image G is zoomed out while the specific point P being maintained in the center.

In step S5, in addition to or instead of the movement of the endoscope 2, the processor 5a may control the zoom lens 2b of the endoscope 2 to thereby optically zoom out the image G.

After starting the zoom-out, the processor 5a determines an end trigger for ending the zoom-out (step S7).

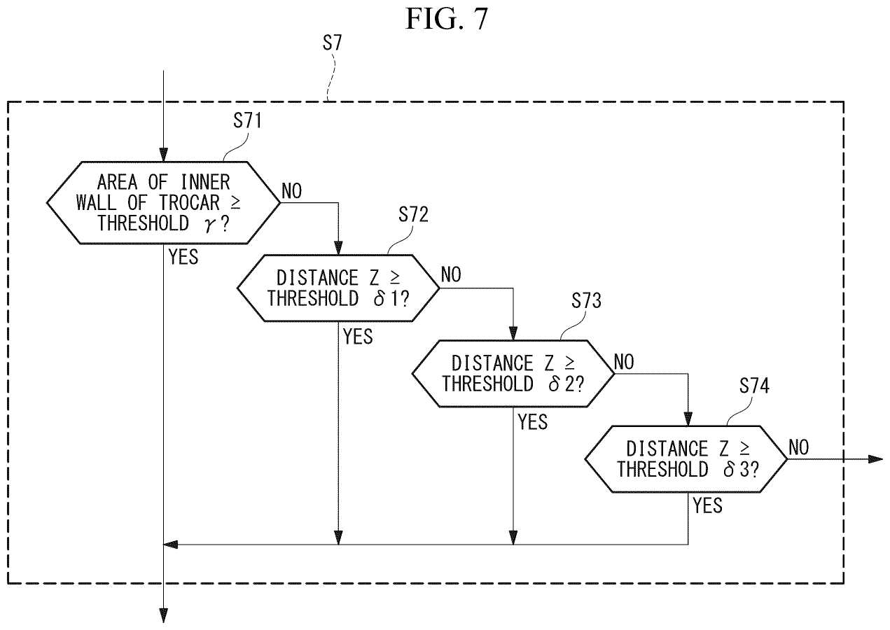

As illustrated in FIG. 7, step S7 includes step S71 of determining the end trigger based on the image G and steps S72, S73, and S74 of determining the end trigger based on a distance Z from the specific point P to the distal end 2c of the endoscope 2. The processor 5a repeats steps S71, S72, S73, and S74 until determining in any one of steps S71, S72, S73, and S74 that the end trigger is ON. When determining in any one of steps S71, S72, S73, and S74 that the end trigger is ON (YES in step S7), the processor 5a stops the endoscope 2 and/or the zoom lens 2b to thereby end the zoom-out (step S8).

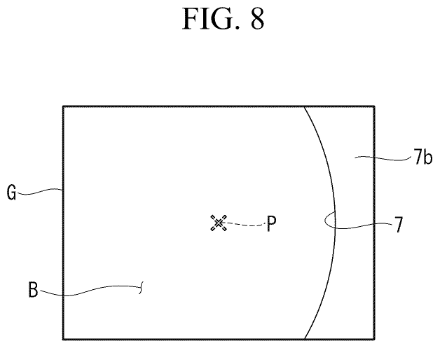

As illustrated in FIG. 8, when the distal end 2c of the endoscope 2 has retracted to the vicinity of a distal end 7a of the trocar 7, an inner wall 7b of the trocar 7 is reflected in the image G. The processor 5a recognizes the inner wall 7b of the trocar 7 in the image G during the zoom-out, calculates the area of the inner wall 7b, and, when the area of the inner wall 7b has reached a predetermined area threshold y, determines that the end trigger is ON (YES in step S71). The threshold y is, for example, an area equivalent to a predetermined percentage of the total area of the image G.

During the zoom-out, the processor 5a calculates the distance Z in the direction along the optical axis from the specific point P to the distal end 2c of the endoscope 2 and, when the distance Z has reached a predetermined distance threshold, determines that the end trigger is ON. Specifically, the predetermined distance threshold includes a first threshold 81, a second threshold 82, and a third threshold 83. When the distance Z has reached any one of the three thresholds 81, 82, and 83, the processor 5a determines that the end trigger is ON (YES in step S72, YES in step S73, or YES in step S74).

The first threshold 81 specifies a condition for the distance Z for acquiring the image G having resolution sufficient for observation of a target site after the zoom-out ends and is determined based on a limit far point of the depth of field of the endoscope 2. For example, the first threshold 81 is the distance from the distal end 2c of the endoscope 2 to the limit far point.

The second threshold 82 specifies a condition for a distance for the treatment instrument 6 reinserted to the specific point P to be able to be followed and is determined based on the resolution of the image G. For example, the second threshold 82 is a limit of the distance Z at which the image G has resolution capable of recognizing an image of the treatment instrument 6 disposed at the specific point P.

Like the second threshold 82, the third threshold 83 specifies a condition for the distance Z for the treatment instrument 6 reinserted to the specific point P to be able to be followed and is determined based on the accuracy of stereoscopic measurement. For example, the third threshold 83 is a limit of a distance at which a three-dimensional position of the treatment instrument 6 disposed at the specific point P can be stereoscopically measured at predetermined accuracy from the image G.

After removing the treatment instrument 6, as illustrated in FIG. 3D, the user reinserts the treatment instrument 6 into the subject A through the trocar 8 and moves the treatment instrument 6 toward a target site.

After the zoom-out ends, the processor 5a determines, based on the treatment instrument information, a return trigger indicating that the treatment instrument 6 has been reinserted (step S9) and ends the overlooking mode in response to the return trigger being turned on (step S10).

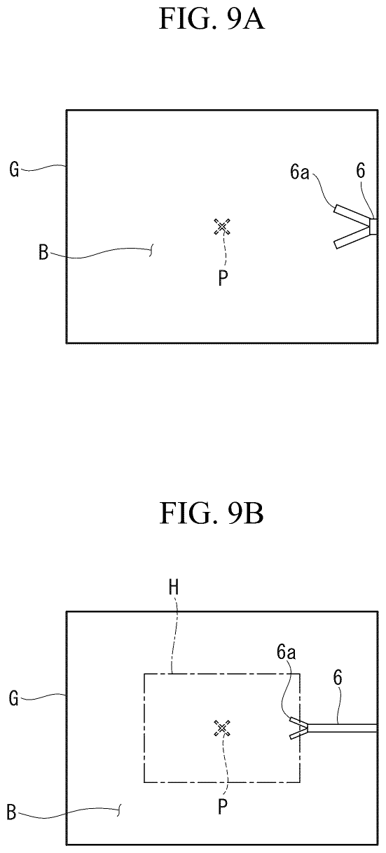

FIG. 9A and FIG. 9B explain an example of the determination of the return trigger.

In a first example illustrated in FIG. 9A, the treatment instrument information is presence or absence of the treatment instrument 6 in the image G. The processor 5a detects, as the return trigger, presence of the treatment instrument 6 in the image G. That is, when the treatment instrument 6 has not been recognized in the image G, the processor 5a determines that the return trigger is OFF (NO in step S9). When the treatment instrument 6 has been recognized in the image G, the processor 5a determines that the return trigger is ON (YES in step S9).

In a second example illustrated in FIG. 9B, the treatment instrument information is presence or absence of the treatment instrument 6 in a predetermined region H in the image G. The processor 5a detects, as the return trigger, presence of the treatment instrument 6 in the predetermined region H. The predetermined region H is a portion of the image G including the specific point P and is, for example, a center region of the image G. That is, when the treatment instrument 6 has not been recognized in the predetermined region H, the processor 5a determines that the return trigger is OFF (NO in step S9). When the treatment instrument 6 has been recognized in the predetermined region H, the processor 5a determines that the return trigger is ON (YES in step S9).

According to the second example, during zoom-in performed thereafter, it is possible to continuously capture, in the center of the image G, a target site observed immediately before the manual mode or the following mode is switched to the overlooking mode.

The treatment instrument 6 can be reinserted before the zoom-out ends. Therefore, the processor 5a may determine the return trigger between step S5 and step S7 in addition to after step S8 (step S6).

When determining that the return trigger is ON (YES in step S6 or S9), the processor 5a switches the overlooking mode to the following mode or the manual mode in step S1 (step S10).

When the overlooking mode has been switched to the following mode, the processor 5a recognizes the treatment instrument 6 in the zoomed-out image G and subsequently controls the moving device 3 to thereby move the endoscope 2 to a position where the target point coincides with the distal end 6a. Accordingly, the image G automatically zooms in and the position of the endoscope 2 in the subject A and the magnification of the image G return to the states before the overlooking mode (see FIG. 3A and FIG. 4A).

When the overlooking mode has been switched to the manual mode, for example, the processor 5a controls the moving device 3 to thereby move the endoscope 2 and, at a point in time when it is determined that the start trigger is ON, moves the distal end 2c to a position where the distal end 2c is disposed. Accordingly, the image G automatically zooms in.

As explained above, according to the present embodiment, the removal of the treatment instrument 6 is automatically detected based on the treatment instrument information, the overlooking mode is automatically execute after the removal of the treatment instrument 6, and the image G automatically zooms out. In a zoomed-out state, the user can easily reinsert the treatment instrument 6 to the target site while observing the image G in a wide range in the subject A. In this way, it is possible to support the easy reinsertion of the treatment instrument 6 without requiring operation of the user.

In order to observe the wide range in the subject A, the magnification of the zoomed-out image G is preferably lower. On the other hand, when the image G excessively zooms out, for example, when the distance Z is excessively large, problems can occur in the observation, the image recognition, and the following of the reinserted treatment instrument 6. According to the present embodiment, in steps S71 to S74, the position of the endoscope 2 that ends the zoom-out is automatically determined, based on the image G and the distance Z, in a position where the magnification is the lowest in a range in which satisfactory observation and satisfactory following of the reinserted treatment instrument 6 are guaranteed. Accordingly, it is possible to more effectively support the reinsertion of the treatment instrument 6.

The reinsertion of the treatment instrument 6 is automatically detected based on the treatment instrument information. The endoscope 2 automatically returns to the original mode after the reinsertion of the treatment instrument 6. Accordingly, it is possible to make operation of the user for switching the overlooking mode to the following or manual mode unnecessary and more effectively support the reinsertion of the treatment instrument 6.

In the present embodiment, as illustrated in FIG. 3E, the processor 5a may move the visual field F of the endoscope 2 toward a distal end 8a of the trocar 8 in parallel to or after the zoom-out of the image G.

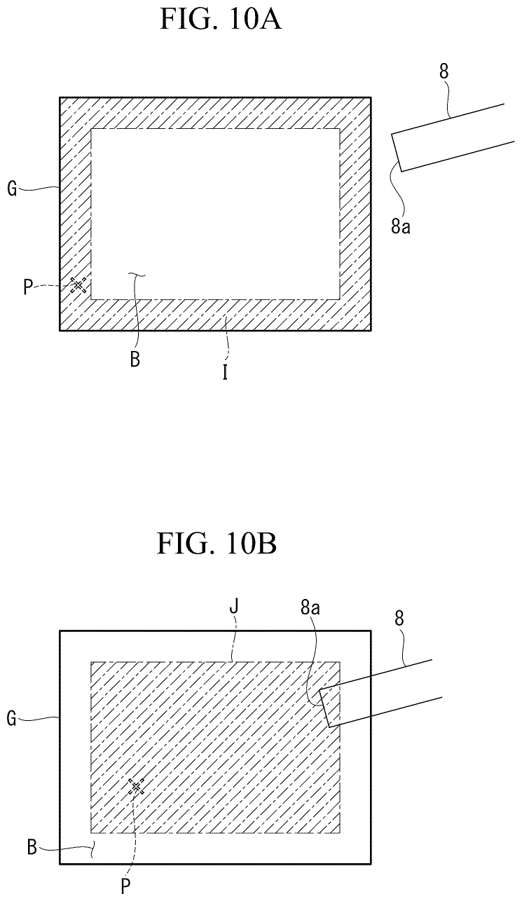

FIG. 3E illustrates a case in which the visual field F is moved after the zoom-out. When the area of the inner wall 7b has reached the threshold y or the distance Z has reached any one of the thresholds 81, 82, and 83 (YES in step S71, S72, S73, or S74), the processor 5a controls the moving device 3 to thereby swing the endoscope 2 in a direction in which the distal end 2c of the endoscope 2 approaches the distal end 8a of the trocar 8. Accordingly, as illustrated in FIG. 10A and FIG. 10B, the specific point P moves from the center to the end in the image G.

Specifically, the processor 5a calculates a position coordinate of the distal end 8a of the trocar 8 from a position coordinate of a pivot point D of the trocar 8 and an insertion length L3 of the trocar 8 and swings the endoscope 2 toward the distal end 8a. The insertion length L3 is the length of the trocar 8 from the distal end 8a to the pivot point D. The position coordinates of the pivot point D and the distal end 8a are coordinates in the world coordinate system. Here, it is assumed that the distal end 8a faces the specific point P.

The processor 5a moves the visual field F toward the distal end 8a until the specific point P is present in the image G and, if possible, the distal end 8a of the trocar 8 is reflected in the image G.

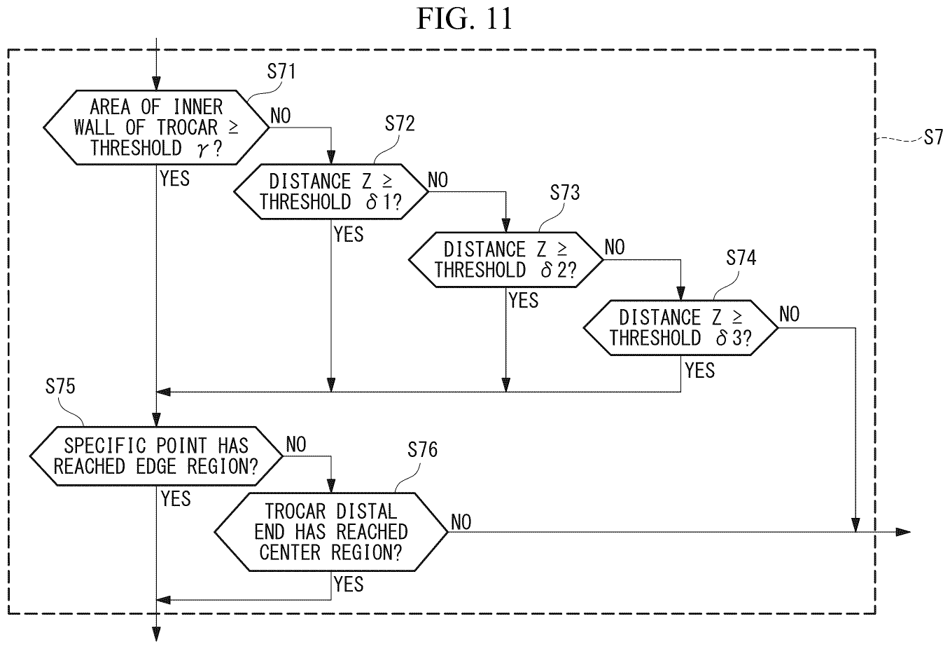

Specifically, as illustrated in FIG. 11, the processor 5a determines whether the specific point P has reached a peripheral edge region I of the image G (step S75) and whether the distal end 8a of the trocar 8 has reached a center region J of the image G (step S76). The peripheral edge region I is a region having a predetermined width and extending along the edge of the image G (see FIG. 10A). The center region J is a portion of the image G including the center of the image G (see FIG. 10B). When the specific point P has reached the peripheral edge region I (YES in step S75) or the distal end 8a has reached the center region J (YES in step S76), the processor 5a determines that the end trigger is ON.

When moving the visual field F in parallel to the zoom-out, the processor 5a swings the endoscope 2 while retracting the endoscope 2 and performs steps S75 and S76 in parallel to steps S71 to S74.

According to modifications illustrated in FIG. 10A to FIG. 11, after the zoom-out, the visual field F of the endoscope 2 is brought close to the distal end 8a of the trocar 8 in a range in which the specific point P is maintained in the image G. Both of the specific point P and the distal end 8a are preferably included in the image G. Accordingly, the user can more easily observe the treatment instrument 6 to be reinserted. It is possible to more effectively support the reinsertion of the treatment instrument 6.

When the endoscope 2 includes a mechanism that changes the direction of the visual field F, the control device 5 may control the endoscope 2 to thereby move the visual field F toward the distal end 8a. The mechanism is, for example, a curved portion provided at the distal end portion of the endoscope 2.

In the embodiment explained above, step S7 for determining the end trigger includes the four steps S71, S72, S73, and S74. However, step S7 only has to include at least one of steps S71, S72, S73, and S74. For example, when the resolution of the image G and the accuracy of the stereoscopic measurement are sufficiently high, step S7 may include only step S71.

In the embodiment explained above, the processor 5a switches the overlooking mode to the same mode as the mode immediately preceding the overlooking mode. However, instead of this, the processor 5a may switch the overlooking mode to a predetermined mode.

For example, the control device 5 may be configured such that the user can set a mode after the overlooking mode to one of the manual mode and the following mode. In this case, regardless of the mode immediately preceding the overlooking mode, the processor 5a may switch the overlooking mode to a mode set in advance by the user.

In step S3 in the embodiment explained above, the processor 5a determines the start trigger based on one piece of treatment instrument information. However, instead of this, the processor 5a may determine the start trigger based on a combination of two or more pieces of treatment instrument information.

That is, the processor 5a may acquire two or more pieces of treatment instrument information in step S2 and, thereafter, execute two or more of the first to fourth methods illustrated in FIG. 6A to FIG. 6D. For example, when two or more of the length of the disappearance time, the speed of the treatment instrument 6, and the path of the treatment instrument 6 have exceeded the thresholds corresponding thereto, the processor 5a may determine that the start trigger is ON and start the overlooking mode.

In the embodiment explained above, the processor 5a automatically detects the start trigger. However, instead of or in addition to this, the processor 5a may set input of the user as the start trigger.

For example, the user can input the start trigger to the control device 5 at any timing using the user interface. The processor 5a responds to the input of the start trigger and executes the overlooking mode. With this configuration, the user can cause the endoscope 2 and the moving device 3 to execute zoom-out of the image G at desired any timing.

Similarly, the processor 5a may end the zoom-out and the overlooking mode respectively in response to the end trigger and the return trigger input to the user interface by the user.

In the embodiment explained above, the control device 5 is the endoscope processor. However, instead of this, the control device 5 may be any device including the processor 5a and the recording medium 5c storing the control program 5e. For example, the control device 5 may be incorporated in the moving device 3 or may be any computer such as a personal computer connected to the endoscope 2 and the moving device 3.

The embodiment and the modifications of the present disclosure are explained in detail above. However, the present disclosure is not limited to the embodiment and the modifications explained above. Various additions, substitutions, changes, partial deletions, and the like of the embodiment and the modifications are possible without departing from the gist of the disclosure or without departing from the idea and the meaning of the present disclosure derived from the content described in the claims and equivalents of the claims.

### REFERENCE SIGNS LIST

1 Endoscope system
2 Endoscope
2a Image pickup element
3 Moving device (Robot arm)
4 Display device
5 Control device (Controller)
5a Processor
5c Storage unit (Recording medium)
5e Control program
6 Treatment instrument
6a Distal end
7, 8 Trocar
7a Distal end
7b Inner wall
9 Sensor
A Subject
G Image
F Specific point

1. An endoscope system comprising:
an endoscope to be inserted into a subject to acquire an image of an inside of the subject;
a robot arm configured to change a position and a posture of the endoscope; and
a controller comprising at least one processor,
wherein the controller is configured to:
acquire treatment instrument information concerning a position or a movement of a treatment instrument to be inserted into the subject,
determine whether the treatment instrument has been removed based on the treatment instrument information,
in response to determining that the treatment instrument has been removed, execute an overlooking mode, and
in the overlooking mode, control at least one of the endoscope or the robot arm to thereby automatically zoom out the image while maintaining, in the image, a specific point in the subject.

2. The endoscope system according to claim 1, wherein the controller is configured to start and end the overlooking mode based on the treatment instrument information.

3. The endoscope system according to claim 1, wherein the controller is configured to control the robot arm and move the endoscope in a direction away from the specific point to thereby zoom out the image.

4. The endoscope system according to claim 1, wherein the controller is configured to control the endoscope to optically zoom out the image.

5. The endoscope system according to claim 1, wherein
the controller is configured to detect the treatment instrument information from the image acquired by the endoscope.

6. The endoscope system according to claim 1, further comprising a sensor configured to detect the position of the treatment instrument.

7. The endoscope system according to claim 1, wherein
the controller is configured to switch a manual mode to the overlooking mode and/or switch the overlooking mode to the manual mode, and
in the manual mode, the controller is configured to permit operation of the endoscope by a user.

8. The endoscope system according to claim 1, wherein
the controller is configured to switch a following mode to the overlooking mode and/or switch
the overlooking mode to the following mode, and
in the following mode, the controller is configured to control the robot arm based on the position of the treatment instrument to thereby cause the endoscope to follow the treatment instrument.

9. The endoscope system according to claim 1, wherein, in the overlooking mode, the controller is configured to control the at least one of the endoscope or the robot arm to thereby move a visual field of the endoscope toward a distal end of a trocar through which the treatment instrument pierces.

10. The endoscope system according to claim 9, wherein the controller is configured to end the movement of the visual field in response to the specific point having reached a peripheral edge region in the image or in response to the distal end of the trocar through which the treatment instrument pierces having reached a center region in the image.

11. The endoscope system according to claim 1, wherein the treatment instrument information includes any one of presence or absence of the treatment instrument in the image, length of a disappearance time in which the treatment instrument is continuously absent in the image, a position, speed, and a path of the treatment instrument, and a combination thereof.

12. The endoscope system according to claim 11, wherein
the treatment instrument information is any one of the length of the disappearance time, the speed of the treatment instrument and the path of the treatment instrument, and a combination thereof, and
the controller is configured to start the overlooking mode in response to the treatment instrument information having exceeded a predetermined threshold.

13. The endoscope system according to claim 1, wherein
the treatment instrument information includes presence or absence of the treatment instrument in the image, and
in response to the presence of the treatment instrument being detected in the image, the controller is configured to end the overlooking mode.

14. The endoscope system according to claim 1, wherein the controller ends the zooming out of the image when an area of a trocar in the image has reached a predetermined area threshold.

15. The endoscope system according to claim 1, wherein the controller is configured to end the zooming out of the image in response to a distance from the specific point to a distal end of the endoscope having reached a predetermined distance threshold.

16. The endoscope system according to claim 15, wherein the predetermined distance threshold includes at least one of a first threshold determined based on a far point of a depth of field of the endoscope, a second threshold determined based on resolution of the image, and a third threshold determined based on accuracy of stereoscopic measurement of the endoscope.

17. A method for controlling an endoscope system, the endoscope system comprising an endoscope to be inserted into a subject to acquire an image of an inside of the subject and a robot arm configured to change a position and a posture of the endoscope,
the control method comprising:
acquiring treatment instrument information concerning a position or a movement of a treatment instrument inserted into the subject;
determining whether the treatment instrument has been removed based on the treatment instrument information;
in response to determining that the treatment instrument has been removed, executing an overlooking mode; and
in the overlooking mode, controlling at least one of the endoscope or the robot arm to thereby automatically zoom out the image while maintaining, in the image, a specific point in the subject.

18. A non-transitory computer-readable recording medium storing a control program for causing a computer to execute the control method according to claim 17.
